(19) United States
(12) Patent Application Publication
(10) Pub. No.: US 2001/0053776 A1
(43) Pub. Date: Dec. 20, 2001

(54) Tricyclic pyridin-2-one analogue as a GABA receptor ligand

(76) Inventors: James Michael Crawforth, Stevenage (GB); Karl Richard Gibson, Bishops Stortford (GB); Michael Rowley, Casalpolocco (IT)

Correspondence Address: MERCK AND CO INC, P O BOX 2000, RAHWAY, NJ, 070650907

(21) Appl. No.: 09/861,318

(22) Filed: May 18, 2001

(30) Foreign Application Priority Data
May 24, 2000 (GB) 0012708.4
Feb. 13, 2001 (GB) 0103525.2

(51) Int. Cl. A61K 31/55; C07D 487/14

(52) U.S. Cl. 514/214.01; 540/579

(57) ABSTRACT

9-(4-Methylthiazol-2-yl)- 11-(pyridin-4-yl)-6,7-dihydro-5H-2, 7a-diazadibenzo[a,c]cyclohepten-8-one, and pharmaceutically acceptable salts thereof, are selective ligands for GABAA receptors, in particular having high affinity for the a2 and/or a3 subunit thereof, and are accordingly of benefit in the treatment and/or prevention of disorders of the central nervous system, including anxiety and convulsions.

The present invention relates to a fused tricyclic compound based on a substituted pyridone ring, and to its use in therapy. More particularly, this invention is concerned with a particular tricyclic pyridin-2-one analogue which is a GABAA receptor ligand and is therefore useful in the therapy of deleterious mental states.

Receptors for the major inhibitory neurotransmitter, gamma-aminobutyric acid (GABA), are divided into two main classes: (1) GABA_{A} receptors, which are members of the ligand-gated ion channel superfamily; and (2) GABA_{B} receptors, which may be members of the G-protein linked receptor superfamily. Since the first cDNAs encoding individual GABA_{A} receptor subunits were cloned the number of known members of the mammalian family has grown to include at least six α subunits, four β subunits, three y subunits, one δ subunit, one ε subunit and two ρ subunits.

Although knowledge of the diversity of the GABA_{A} receptor gene family represents a huge step forward in our understanding of this ligand-gated ion channel, insight into the extent of subtype diversity is still at an early stage. It has been indicated that an α subunit, a β subunit and a y subunit constitute the minimum requirement for forming a fully functional GABA_{A} receptor expressed by transiently transfecting cDNAs into cells. As indicated above, δ, ε and ρ subunits also exist, but are present only to a minor extent in GABA_{A} receptor populations.

Studies of receptor size and visualisation by electron microscopy conclude that, like other members of the ligand-gated ion channel family, the native GABA_{A} receptor exists in pentameric form. The selection of at least one α, one β and one γ subunit from a repertoire of seventeen allows for the possible existence of more than 10,000 pentameric subunit combinations. Moreover, this calculation overlooks the additional permutations that would be possible if the arrangement of subunits around the ion channel had no constraints (i.e. there could be 120 possible variants for a receptor composed of five different subunits).

Receptor subtype assemblies which do exist include, amongst many others, α1β2γ2, α2β2/3, α3βγ2, α2βγ1, α5β3γ2/3, α6βγ2, α6βδ and α4βδ. Subtype assemblies containing an α1 subunit are present in most areas of the brain and are thought to account for over 40% of GABA_{A} receptors in the rat. Subtype assemblies containing α2 and α3 subunits respectively are thought to account for about 25% and 17% of GABA_{A} receptors in the rat. Subtype assemblies containing an α5 subunit are expressed predominantly in the hippocampus and cortex and are thought to represent about 4% of GABA_{A} receptors in the rat.

A characteristic property of all known GABA_{A} receptors is the presence of a number of modulatory sites, one of which is the benzodiazepine (BZ) binding site. The BZ binding site is the most explored of the GABA_{A} receptor modulatory sites, and is the site through which anxiolytic drugs such as diazepam and temazepam exert their effect. Before the cloning of the GABA_{A} receptor gene family, the benzodiazepine binding site was historically subdivided into two subtypes, BZ1 and BZ2, on the basis of radioligand binding studies. The BZ1 subtype has been shown to be pharmacologically equivalent to a GABA_{A} receptor comprising the α1 subunit in combination with a β subunit and γ2. This is the most abundant GABA_{A} receptor subtype, and is believed to represent almost half of all GABA_{A} receptors in the brain.

Two other major populations are the α2βγ2 and α3βγ2/3 subtypes. Together these constitute approximately a further 35% of the total GABA_{A} receptor repertoire. Pharmacologically this combination appears to be equivalent to the BZ2 subtype as defined previously by radioligand binding, although the BZ2 subtype may also include certain α5-containing subtype assemblies. The physiological role of these subtypes has hitherto been unclear because no sufficiently selective agonists or antagonists were known.

It is now believed that agents acting as BZ agonists at α1βγ2, α2βγ2 or α3βγ2 subunits will possess desirable anxiolytic properties. Compounds which are modulators of the benzodiazepine binding site of the GABA_{A} receptor by acting as BZ agonists are referred to hereinafter as “GABA_{A} receptor agonists”. The α1-selective GABA_{A} receptor agonists alpidem and zolpidem are clinically prescribed as hypnotic agents, suggesting that at least some of the sedation associated with known anxiolytic drugs which act at the BZ1 binding site is mediated through GABA_{A} receptors containing the α1 subunit. Accordingly, it is considered that GABA_{A} receptor agonists which interact more favourably with the α2 and/or α3 subunit than with α1 will be effective in the treatment of anxiety with a reduced propensity to cause sedation. Also, agents which are antagonists or inverse agonists at α1 might be employed to reverse sedation or hypnosis caused by α1 agonists.

The compounds of the present invention, being selective ligands for GABA_{A} receptors, are therefore of use in the treatment and/or prevention of a variety of disorders of the central nervous system. Such disorders include anxiety disorders, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; and disorders of circadian rhythm, e.g. in subjects suffering from the effects of jet lag or shift work.

Further disorders for which selective ligands for GABA_{A} receptors may be of benefit include pain and nociception; emesis, including acute, delayed and anticipatory emesis, in particular emesis induced by chemotherapy or radiation, as well as post-operative nausea and vomiting; eating disorders including anorexia nervosa and bulimia nervosa; premenstrual syndrome; muscle spasm or spasticity, e.g. in paraplegic patients; and hearing loss. Selective ligands for GABA_{A} receptors may also be effective as pre-medication prior to anaesthesia or minor procedures such as endoscopy, including gastric endoscopy.

WO 98/50384 describes a class of tricyclic pyridin-2-one analogues, substituted at the 3-position of the pyridone ring by an ester or thiazole moiety, which are stated to be selective ligands for GABA_{A} receptors beneficial in the treatment and/or prevention of neurological disorders, including anxiety and convulsions.

The present invention provides a particular tricyclic pyridin-2-one analogue, and pharmaceutically acceptable salts thereof, which possess desirable binding properties at various GABA_{A} receptor subtypes. The compounds in accordance with the present invention have good affinity as ligands for the α2 and/or α3 subunit of the human GABA_{A} receptor. The compounds of this invention interact more favourably with the α2 and/or α3 subunit than with the α1 subunit. Indeed, the compounds of the invention exhibit functional selectivity in terms of a selective efficacy for the α2 and/or α3 subunit relative to the α1 subunit.

The compounds of the present invention are GABA_{A} receptor subtype ligands having a binding affinity (Kᵢ) for the α2 and/or α3 subunit, as measured in the assay described hereinbelow, of less than 1 nM. Furthermore, the compounds in accordance with this invention exhibit functional selectivity in terms of a selective efficacy for the α2 and/or α3 subunit relative to the α1 subunit. Moreover, the compounds according to the present invention possess interesting pharmacokinetic properties, notably in terms of improved oral bioavailability.

The present invention provides 9-(4-methylthiazol-2-yl) 11- (pyridin-4-yl)-6,7-dihydro-5H-2,7a-diazadibenzo[a,c]cyclohepten-8-one of formula I:

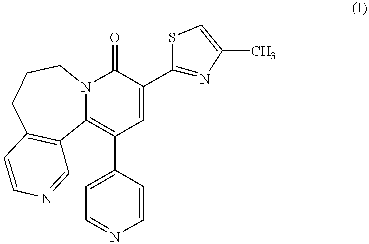

or a pharmaceutically acceptable salt thereof.

The compounds in accordance with the present invention are encompassed within the generic scope of WO 98/50384. There is, however, no specific disclosure therein of the compound of formula I as depicted above, or pharmaceutically acceptable salts thereof

For use in medicine, the salts of the compound of formula I above will be pharmaceutically acceptable salts. Other salts may, however, be useful in the preparation of the compound of formula I or of its pharmaceutically acceptable salts. Suitable pharmaceutically acceptable salts of the compound of formula I include acid addition salts which may, for example, be formed by mixing a solution of the compound of formula I with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid. A favoured class of acid addition salts of the compound of formula I, which possess advantageous properties notably in terms of enhanced solubility, comprises the methanesulfonate salts, in particular the bismethanesulfonate salt. An alternative class of acid addition salts of the compound of formula I, also displaying enhanced solubility, comprises the hydrochloride salts, in particular the bishydrochloride salt.

The fused tricyclic ring system in the compound of formula I as depicted above is asymmetric and is flexible to a certain extent, and the molecule is accordingly likely to exhibit the phenomenon of atropisomerism. Isolation of the individual atropisomers will in principle be possible by the formation of acid addition salts with chiral acids in accordance with standard techniques. It is to be understood that all such stereoisomers, and mixtures thereof in any proportion, are encompassed within the scope of the present invention.

Also provided by the present invention is a method for the treatment and/or prevention of anxiety which comprises administering to a patient in need of such treatment an effective amount of the compound of formula I as depicted above or a pharmaceutically acceptable salt thereof.

Further provided by the present invention is a method for the treatment and/or prevention of convulsions (e.g. in a patient suffering from epilepsy or a related disorder) which comprises administering to a patient in need of such treatment an effective amount of the compound of formula I as depicted above or a pharmaceutically acceptable salt thereof The binding affinity (Kᵢ) of the compounds according to the present invention for the α3 subunit of the human GABA_{A} receptor is conveniently as measured in the assay described hereinbelow. The α3 subunit binding affinity (Kᵢ) of the compounds of the invention is less than 1 nM.

The compounds according to the present invention elicit a selective potentiation of the GABA EC₂₀ response in stably transfected recombinant cell lines expressing the α3 subunit of the human GABA_{A} receptor relative to the potentiation of the GABA EC₂₀ response elicited in stably transfected recombinant cell lines expressing the α1 subunit of the human GABA_{A} receptor.

The potentiation of the GABA EC₂₀ response in stably transfected cell lines expressing the α3 and α1 subunits of the human GABA_{A} receptor can conveniently be measured by procedures analogous to the protocol described in Wafford et al., Mol. Pharmacol., 1996, 50, 670-678. The procedure will suitably be carried out utilising cultures of stably transfected eukaryotic cells, typically of stably transfected mouse Ltk fibroblast cells.

The compounds according to the present invention exhibit anxiolytic activity, as demonstrated by a positive response in the elevated plus maze and conditioned suppression of drinking tests (cf. Dawson et al., Psychopharmacology, 1995, 121, 109-117). Moreover, the compounds of the invention are substantially non-sedating, as confirmed by an appropriate result obtained from the response sensitivity (chain-pulling) test (cf. Bayley et al., J. Psychopharmacol., 1996, 10, 206-213).

The compounds according to the present invention may also exhibit anticonvulsant activity. This can be demonstrated by the ability to block pentylenetetrazole-induced seizures in rats and mice, following a protocol analogous to that described by Bristow et al. in J. Pharmacol. Exp. Ther., 1996, 279, 492-501.

Since they elicit behavioural effects, the compounds of the invention plainly are brain-penetrant; in other words, these compounds are capable of crossing the so-called “blood-brain barrier”. Advantageously, the compounds of the invention are capable of exerting their beneficial therapeutic action following administration by the oral route.

The invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of anxiety, a suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day.

The compound of formula I as depicted above may be prepared by a process which comprises cyclising a compound of formula II:

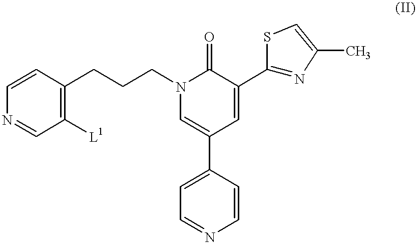

wherein L¹ represents a readily displaceable group.

The readily displaceable group L¹ is suitably a halogen atom, e.g. bromo, in which case the cyclisation is conveniently carried out by treating the compound of formula II with tributyltin hydride in the presence of a radical initiator such as 1,1′-azobisisobutyronitrile (AIBN), typically in an inert solvent such as benzene.

In an alternative procedure, the compound of formula I as depicted above may be prepared by a process which comprises cyclising a compound of formula III:

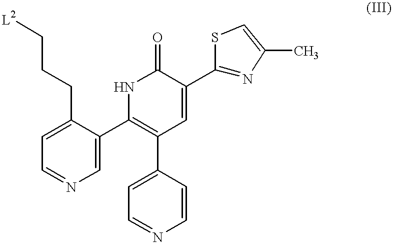

wherein L² represents a readily displaceable group.

The readily displaceable group L² may suitably be a halogen atom, e.g. bromo, in which case the cyclisation of compound III is conveniently effected by treatment with sodium hydride in the presence of lithium bromide, in a solvent system which may typically be a mixture of 1,2-dimethoxyethane and N,N-dimethylformamide. Alternatively, the readily displaceable group L² may be hydroxy, in which case the cyclisation of compound III is conveniently effected by treatment with triphenylphosphine in the presence of diethyl azodicarboxylate (DEAD), typically in an inert solvent such as tetrahydrofuran or dichloromethane.

The intermediates of formula II above may suitably be prepared by reacting a compound of formula IV with the compound of formula V:

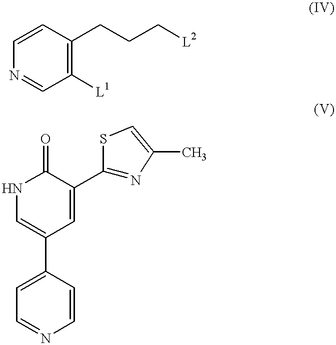

wherein L¹ and L² are as defined above; under conditions analogous to those described above for the cyclisation of compound III.

In another procedure, the compound of formula I as depicted above may be prepared by a process which comprises reacting a compound of formula VI with a compound of formula VII:

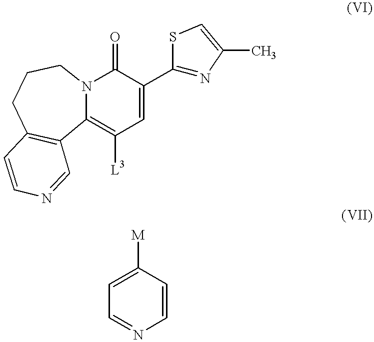

wherein M represents —B(OH)₂ or —Sn(Alk)₃ in which Alk represents a C₁₆ alkyl group, typically n-butyl, and L³ represents a suitable leaving group; in the presence of a transition metal catalyst.

The leaving group L³ is suitably a halogen atom, e.g. bromo; or a sulfonyloxy moiety, e.g. methanesulfonyloxy (mesyloxy), trifluoromethanesulfonyloxy (triflyloxy) or p-toluenesulfonyloxy (tosyloxy). Favourably, L³ represents triflyloxy.

A suitable transition metal catalyst of use in the reaction between compounds VI and VII comprises dichlorobis(triphenylphosphine)-palladium(II) or tetrakis(triphenylphosphine)palladium(0).

The reaction between compounds VI and VII is conveniently effected in an inert solvent such as N,N-dimethylformamide, typically in the presence of potassium phosphate at an elevated temperature.

The preparation of the intermediate of formula VI as depicted above wherein L³ represents triflyloxy is described in WO 98/50384.

Where they are not commercially available, the starting materials of formula III, IV, V and VII may be prepared by methods analogous to those described in the accompanying Examples, or by standard methods well known from the art.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The following Examples illustrate the preparation of compounds according to the invention.

The compounds in accordance with this invention potently inhibit the binding of [³H]-flumazenil to the benzodiazepine binding site of human GABA_{A} receptors containing the α2 or α3 subunit stably expressed in Ltk-cells.

Reagents

Phosphate buffered saline (PBS).

Assay buffer: 10 mM KH₂PO₄, 100 mM KCl, pH 7.4 at room temperature.

[³]-Flumazenil (18 nM for α1β3γ2 cells; 18 nM for α2β3γ2 cells; 10 nM for α3β3γ2 cells) in assay buffer.

Flunitrazepam 100 μM in assay buffer.

Cells resuspended in assay buffer (1 tray to 10 ml).

Harvesting Cells

Supernatant is removed from cells. PBS (approximately 20 ml) is added. The cells are scraped and placed in a 50 ml centrifuge tube. The procedure is repeated with a further 10 ml of PBS to ensure that most of the cells are removed. The cells are pelleted by centrifuging for 20 min at 3000 rpm in a benchtop centrifuge, and then frozen if desired. The pellets are resuspended in 10 ml of buffer per tray (25 cm×25 cm) of cells.

Assay

Can be carried out in deep 96-well plates or in tubes. Each tube contains:

300 μl of assay buffer.

50 μl of [³H]-flumazenil (final concentration for α1β3γ2: 1.8 nM; for α2β3γ2: 1.8 nM; for α3β3γ2: 1.0 nM).

50 μl of buffer or solvent carrier (e.g. 10% DMSO) if compounds are dissolved in 10% DMSO (total); test compound or flunitrazepam (to determine non-specific binding), 10 μM final concentration.

100 μl of cells.

Assays are incubated for 1 hour at 40° C., then filtered using either a Tomtec or Brandel cell harvester onto GF/B filters followed by 3×3 ml washes with ice cold assay buffer. Filters are dried and counted by liquid scintillation counting. Expected values for total binding are 3000-4000 dpm for total counts and less than 200 dpm for non-specific binding if using liquid scintillation counting, or 1500-2000 dpm for total counts and less than 200 dpm for non-specific binding if counting with meltilex solid scintillant. Binding parameters are determined by non-linear least squares regression analysis, from which the inhibition constant Kᵢ can be calculated for each test compound.

The compound of the accompanying Examples was tested in the above assay, and was found to possess a Kᵢ value for displacement of [³H]-flumazenil from the α2 and/or α3 subunit of the human GABA_{A} receptor of less than 1 nM.

### EXAMPLE 1

### 9- (4-Methylthiazol-2-yl)-11- (pyridin-4-yl)-6,7-dihydro-5H-2,7a-diazadibenzo[a,c]cyclohepten-8-one

a) 3-Bromo-4-[3-(tert-butyldimethylsilanyloxy)propyl]pyridine

Imidazole (19.4 g, 285.9 mmol) was added to a solution of 3-bromo-4-(3-hydroxypropyl)pyridine (47.5 g, 219.9 mmol) in dry dichloromethane (250 ml) followed by tert-butyldimethylsilyl chloride (34.8 g, 230.9 mmol) and the reaction mixture stirred at room temperature for 17 hours. The reaction mixture was washed with water (3×200 ml), dried (MgSO₄) and concentrated in vacuo to a colourless oil (72.5 g; 99.8%): ¹H NMR (400 MHz, CDCl₃) δ 0.07 (s, 6H), 0.84 (s, 9H), 1.75-1.78 (m, 2H), 2.71-2.75 (m, 2H), 3.60 (t, J=6, 2H), 7.10 (d, J=5, 1H), 8.32 (d, J=5, iH), 8.57 (s, 1H). m/z (ES⁺) 330, 332 M⁺+H).

b) 4-[3-(tert-Butyldimethvlsilanyloxy)propyl]nicotinic acid methyl ester

A solution of the foregoing product (20.0 g, 60.6 mmol) in dry methanol (250 ml) and DMF (250 ml) was degassed with nitrogen for 20 minutes. Ethyldiisopropylamine (31.7 ml, 181.8 mmol) and 1,3-bis(diphenylphosphino)propane (2.5 g, 6.0 mmol) was added and the solution degassed for a further 5 minutes. Palladium(II) acetate (1.3 g, 6.0 mmol) was added, and carbon monoxide bubbled through for 20 minutes at room temperature. The flow rate was reduced and the mixture heated at 95° C. for 20 hours. The reaction mixture was poured into water (1 1) and extracted with EtOAc (3×400 ml), dried (MgSO₄) and concentrated in vacuo. The residue was purified by flash chromatography eluting with EtOAc:isohexanes (30/70) to yield the title compound as a brown oil (33.5 g, 88%): ¹H NMR (400 MHz, CDCl₃) 6 0.07 (s, 6H), 0.85 (s, 9H), 1.75-1.79 (m, 2H), 2.96-3.00 (m, 2H), 3.60 (t, J=6, 2H), 3.86 (s, 3H), 7.14 (d, J=5, 1H), 8.52 (d, J=5, 1H), 8.98 (s, 1H). m /z (ES+) 310 (M⁺+H).

c) 4-[3-(tert-Butyldimethylsilaniloxypropyl]nicotinic acid

To a suspension of potassium trimethylsilanolate (9.4 g, 72.8 mmol) in dry diethyl ether (250 ml) was added the foregoing ester (15.0 g, 48.5 mmol) and the mixture stirred at room temperature for 48 hours. The resulting white solid was filtered, taken into water (300 ml) and the pH adjusted with citric acid (10% w/v) to pH 5. The aqueous was extracted with EtOAc (2×300 ml), dried (MgSO₄) and evaporated to give the title compound (12.8 g, 89%) as a white solid, m.p. 84-86° C. (from EtOAc): ¹H NMR (400 MHz, d₆-DMSO) δ 0.07 (s, 6H), 0.84 (s, 9H), 1.71-1.78 (m, 2H), 2.93-2.97 (m, 2H), 3.60 (t, J=6, 2H), 7.31 (d, J=5, 1H), 8.55 (d, J=5, 1H), 8.96 (s, 1H), 13.25 (br s, 1H). m/z (ES+) 296 (M⁺+H).

d) 3-f4-[3-(tert-Butyldimethylsilanyloxypropyl]pyridin-3-yR-3-oxo-2-(pyridin-4-yl)propionic acid ethyl ester

N,N′-Carbonyldiimidazole (2.53 g, 15.6 mmol) was added to a stirred solution of 4-[3-(tert-butyldimethylsilanyloxy)propyl]nicotinic acid (4.40 g, 14.9 mmol) in dry DMF (300 ml) under nitrogen. The reaction was heated to 50° C. for 90 minutes and then cooled to −10° C. and ethyl 4-pyridylacetate (2.4 ml, 15.6 mmol) added followed by sodium hydride (2.09 g, 60% dispersion in mineral oil, 52 mmol). The reaction was stirred at −10° C. for 1 hour and allowed to warm to room temperature before stirring for 16 hours. The reaction was quenched by pouring into NH₄Cl solution (750 ml, sat. aqueous). The aqueous was extracted with EtOAc (3×500 ml) and the combined organic extracts were washed with water (2×250 ml), brine (250 ml), dried (Nα2SO₄) and evaporated. The crude product was purified by flash chromatography eluting with EtOAc to afford the title compound (4.40 g, 70%) as a colourless oil: ¹H NMR (400 MHz, CDCl₃, as mixture of ketone and enol tautomers) δ 0.07 (s, 6H), 0.05 (s, 6H), 0.83 (s, 9H), 0.85 (s, 9H), 1.16-1.22 (m, 2×3H), 1.65-1.80 (m, 2×2H), 2.60-2.64 (m, 2H), 2.76-2.80 (m, 2H), 3.59 (t, J=6, 2×2H), 4.15 (q, J=7, 2H), 4.23 (q, J=7, 2H), 5.40 (s, 1H), 6.83-6.85 (m, 2×21), 7.02 (d, J=5, 1H), 7.18 (d, J=5, 1H), 8.11 (s, 1H), 8.27-8.29 (m, 2H), 8.30 (d, J=5, 1H), 8.50 (d, J =5, 1H), 8.81 (s, 1H), 13.47 (s, 1H). m/z (ES⁺) 443 (M⁺+H).

e) 1-{4-[3-(tert-Butyldimethylsilaniloxy)propyl]pyridin-3-yl}-2-(pyridin-4-yl)ethanone

Sodium chloride (220 mg, 3.75 mmol) and distilled water (90 μl, 5.12 mmol) were added to a solution of 3-{4-[3-(tert-butyldimethylsilanyloxy)-propyl]pyridin-3-yl}-3-oxo-2-(pyridin-4-yl)propionic acid ethyl ester (1.51 g, 3.41 mmol) in dry DMSO (30 ml). The flask was fitted with a condenser and flushed with nitrogen. The reaction was lowered into a preheated oil bath at 1500C and stirred for 45 minutes. The reaction was allowed to cool and poured into water (250 ml) and extracted with 1:1 Et₂O/EtOAc (3×100 ml). The combined organic extracts were washed with water (2×75 ml), brine (2×75 ml), dried (Na₂SO₄) and evaporated. The resulting oil was purified by flash chromatography eluting with EtOAc to afford the title compound (1.01 g, 80%) as a colourless oil: ¹H NMR (400 MHz, CDCl₃) δ 0.01 (s, 6H), 0.86 (s, 9H), 1.58-1.69 (m, 2H), 2.77-2.81 (m, 2H), 3.57 (t, J=6, 2H), 4.19 (s, 2H), 7.16-7.28 (m, 6H), 8.50 (d, J=5, 1H), 8.90 (s, 1H). m/z (ES⁺) 370 (M⁺+H).

f) 4-(3-Hydroxypropyl)-5′-(4-methylthiazol-2-yl)-1′H-[3,2′;3′,4″]terpyridin-6′-one

1-{4- [3-(tert-Butyldimethylsilanyloxy)propyl]pyridin-3-yl}-2-(pyridin-4-yl)ethanone (750 mg, 2.02 mmol) was dissolved in DMF dimethyl acetal (10 ml) and stirred for 18 hours at room temperature. The DMF dimethyl acetal was evaporated and azeotroped with dry toluene (2×5 ml). The oil thus produced was dissolved in dry DMF (30 ml) and 4-methylthiazole-2-acetamide (350 mg, 2.22 mmol) and sodium hydride (320 mg, 60% dispersion in mineral oil, 8.08 mmol) were added followed by dry methanol (80 μl, 2.02 mmol) and the reaction heated to 50° C. After stirring at 50° C. for 150 minutes the reaction was allowed to cool to room temperature and poured into aqueous hydrochloric acid (10 ml). The mixture was neutralised after 90 seconds by addition of NaHCO₃ (sat. aq.) and extracted with EtOAc (4×50 ml). The combined organic extracts were washed with water (3×30 ml), brine (40 ml), dried (MgSO₄) and evaporated to 5 ml volume at which point a pale solid precipitated. The solid was collected by filtration and washed with EtOAc to afford the title compound (310 mg, 41%) as a pale green amorphous solid: ¹H NMR (400 MHz, d₆-DMSO) 6 1.36-1.48 (m, 1H), 1.58-1.67 (m, 11), 2.30-2.38 (m, 1H), 2.41-2.52 (m, 1H), 2.45 (s, 3H), 3.28-3.32 (m, 2H), 4.48 (br m, 1H), 7.10-7.11 (m, 2H), 7.30 (d, J=5, 1H), 7.36 (s, 1H), 8.41-8.43 (m, 2H), 8.48 (s, 1H), 8.51 (d, J=5, 1H), 8.53 (s, 1H). m/z (ES⁺) 405 (M⁺+H).

g) 9-(4-Methylthiazol-2-yl)-11-(pyridin-4-yl)-6,7-dihydro-5H-2,7a-diaza-dibenzo[a,c]cyclohepten-8-one

Triphenylphosphine (260 mg, 1.0 mmol) was added to a suspension of 4-(3-hydroxypropyl)-5′-(4-methylthiazol-2-yl)-1′h- [3,2′;3′,4″]terpyridin-6′-one (330 mg, 0.80 mmol) in dry THF (100 ml). Diethyl azodicarboxylate (160 μl, 1.00 mmol) was added dropwise and the reaction stirred at room temperature for 10 minutes. The reaction was poured into water (200 ml) and extracted with EtOAc (3×100 ml). The combined organic extracts were washed with brine (100 ml), dried (MgSO₄) and evaporated to afford a yellow solid. The crude was purified by chromatography (5% MeOH/CH₂Cl₂) and the resulting solid triturated with Et₂O to afford the title compound (262 mg, 84%) as yellow needles, m.p. 300-302° C. Anal. Calc. for C₂₂H₁₈N₄O.0.25H₂O: C, 67.58; H, 4.77; N, 14.33. Found: C, 67.81; H, 4.59; N, 14.41. ¹H NMR (360 MHz, CDCl₃) δ 2.04-2.11 (m, 1H), 2.53 (s, 3H), 2.53-2.64 (m, 1H), 2.75-2.85 (m, 1H), 2.92 (dd, J=13 and 7, 1H), 3.12 (td, J=14 and 5, 1H), 5.32 (dd, J=14 and 5, 1H), 6.93-6.95 (m, 2H), 7.08 (s, 1H), 7.29 (d, J=5, 1H), 8.03 (s, 1H), 8.47-8.49 (m, 2H), 8.56 (d, J=5, 1H), 8.70 (s, 1H). m/z (ES⁺) 387 (M⁺+H).

### EXAMPLE 2

### 9-(4-Methylthiazol-2-yl)-8-oxo-11-(Pyridin-4-yl)-5,6,7, 8-tetrahvdro-7a-aza-2-azoniadibenzo[a,c]cycloheptene, bismethanesulfonate

Methanesulfonic acid (88 μl, 1.36 mmol) was added to a hot solution of 9-(4-methylthiazol-2-yl)-11-(pyridin-4-yl)-6,7-dihydro-5H-2,7a-diaza-dibenzo[a,c]cyclohepten-8-one (260 mg, 0.66 mmol) in EtOAc (20 ml) and EtOH (100 ml). The mixture was allowed to cool and upon addition of Et₂O a yellow solid crystallised. The solid was collected by filtration and washed with Et₂O and dried under vacuum to afford the title salt (372 mg, 97%) as yellow needles, m.p. 294-297° C. Anal. Calc. for C₂₄H₂₆N₄O₇S₂.H₂O: C, 48.31; H, 4.73; N, 9.39. Found: C, 48.17; H, 4.75; N, 9.15. 1H NMR (400 MHz, d₆-DMSO) 5 2.08-2.15 (m, 1H), 2.32 (s, 6H), 2.35-2.45 (m, 1H), 2.45 (s, 3H), 2.95-2.98 (m, 2H), 3.12 (td, J-=14 and 5, 1H), 5.06 (dd, J=14 and 6, 1H), 7.43 (s, 1H), 7.60-7.62 (m, 3H), 8.02 (s, 1H), 8.61 (s, 1H), 8.62 (d, J=6, 2H), 8.71 (d, J=6, 2H). mr/z (ES⁺) 387 (M⁺+H).

### EXAMPLE 3

### 9-(4-Methylthiazol-2-yl)-11-(pyridin-4-yl)-6,7-dihydro-5H-2,7a-diaza-dibenzo[a,c]cylohepten-8-one: Alternative Procedure I

Pyridine-4-boronic acid was added to a solution of trifluoromethanesulfonic acid 9-(4-methylthiazol-2-yl)-8-oxo-5,6,7,8-tetrahydro-2,7a-diazadibenzo[a,c]cyclohepten-11-yl ester (500 mg, 1.09 mmol) and potassium phosphate (400 mg, 1.88 mmol) in dry DMF (20 ml), and the solution degassed for 10 minutes. Tetrakis(triphenylphosphine)-palladium(0) (100 mg, 0.09 mmol) was added and the reaction mixture heated at 100° C. for 7 hours, poured into water (30 ml), extracted with EtOAc (2×30 ml), dried (MgSO₄) and evaporated. The residue was purified by chromatography (5% MeOH/CH₂Cl₂) and the resulting solid triturated with Et₂O to afford the title compound (137 mg, 36%).

### EXAMPLE 4

### 9-(4-Methylthiazol-2-yl)-11-(pvridin-4-yl)-6,7-dihvdro-5H-2,7a-diazadibenzo[a,c]cyclohepten-8-one: Alternative Procedure II

a) Benzyl 4-pyridylacetate

Ethyl 4-pyridylacetate (100 g, 0.61 mol) was dissolved in benzyl alcohol (300 ml, 3.01 mol), and a catalytic amount of potassium carbonate was added. The reaction was heated to 120° C. and nitrogen bubbled through to evaporate ethanol as it was released. After 6 hours the reaction was allowed to cool and was partitioned between 2N HCl (750 ml) and ether (500 ml). The aqueous was washed with ether (3×500 ml) and then neutralised with NaOH at which point turbidity appeared. The aqueous was extracted with EtOAc (3×500 ml) and the combined EtOAc extracts were washed with brine (250 ml), dried (Na₂SO₄) and evaporated to afford the title compound as a waxy solid (103 g, 75%). ¹H NMR (360 MHz, CDCl₃) 8 3.66 (s, 2H), 5.15 (s, 2H), 7.20 (d, J=4, 2H), 7.26-7.38 (m, 5H), 8.54 (d, J=4, 2H). m/z (ES⁺) 318 (M⁺+H).

b) 3-{4- [3-(tert-Butyldimethylsilanyloxypropyl]pyridin-3-yl}-3-oxo-2-(pyridin-4-yl)propionic acid benzyl ester

To a solution of 4-[3-(tert-butyldimethylsilanyloxy)propyl]nicotinic acid (24.2 g, 81.9 mmol) in dry DMF (600 ml) was added N,N′-carbonyldiimidazole (13.95 g, 86 mmol) and the reaction heated to 45° C. for 90 minutes. The reaction was cooled to −10° C. and benzyl 4-pyridylacetate (19.55 g, 86 mmol) was added followed by sodium hydride (11.47 g, 60% dispersion in oil, 287 mmol) in portions over 20 minutes. The reaction was allowed to warm to room temperature and stirred for 2 hours before the reaction was quenched by pouring onto ice/water and the pH adjusted to neutral with citric acid. The aqueous was extracted with EtOAc (4×500 ml) and the combined organic extracts were washed with water (4×250 ml), brine (2×250 ml), dried (Na₂SO₄) and the solvent removed in vacuo. The crude product was purified by flash chromatography (EtOAc) to afford the title compound as an oil (33.5 g, 81%). 1H NMR (400 MHz, CDCl₃, as mixture of ketone and enol tautomers) δ 0.07 (s, 6H), 0.05 (s, 6H), 0.83 (s, 2×9H), 1.60-1.70 (m, 2H), 1.70-1.80 (m, 2H), 2.62-2.66 (m, 2H), 2.76-2.80 (m, 2H), 3.52 (t, J=6, 2H), 3.60 (t, J=6, 2H), 5.12 (s, 2H), 5.22 (s, 211, 5.45 (s, 1H), 6.85 (d, J=5, 211), 7.03 (d, J=5, 1H), 7.19-7.29 (m, 2×7H), 8.12 (s, 1H), 8.28-8.33 (m, 2×2H), 8.81 (s, 1H), 13.47 (s, 1H). m/z (ES⁺) 506, 397 (M⁺+H, M+H—BnOH).

c) 1-{4-[3-(tert-Butyldimethylsilanyloxy)propyl]pyridin-3-yl}-2-(pyridin-4-yl)ethanone

3-{4-[3-(tert-Butyldimethylsilanyloxy)propyl]pyridin-3-yl}-3-oxo-2-(pyridin-4-yl)propionic acid benzyl ester (32.5 g, 64.4 mmol) was dissolved in ethanol (600 ml) in a 3 neck flask and the system flushed with nitrogen. Palladium on carbon catalyst (3.25 g, 10% Pd) was added as a slurry in water (10 ml) (to reduce risk of fire) followed by ammonium formate (20.3 g, 322 mmol). The flask was fitted with a condenser and heated to 60° C. for 30 minutes. The reaction was allowed to cool and then filtered through celite and the filtrate was evaporated in vacuo and the residue partitioned between water (500 ml) and EtOAc (500 ml). The organic layer was washed with brine, dried (Na₂SO₄) and evaporated to afford the product as a colourless oil (21.6 g, 91%) which was used without further purification. 1H NMR (400 MHz, CDC13) δ 0.01 (s, 6H), 0.86 (s, 9H), 1.58-1.69 (m, 2H), 2.77-2.81 (m, 2H), 3.57 (t, J=6, 2H), 4.19 (s, 2H), 7.16-7.28 (m, 6H), 8.50 (d, J=5, 1H), 8.90 (s, 1H). ml/z (ES⁺) 370 (M⁺+H).

d) 9-(4-Methylthiazol-2-yl)-11-(pyridin-4-yl)-6,7-dih dro-5H-2,7a-diaza-dibenzo [a,c]cyclohepten-8-one

Prepared from the foregoing product by the procedures described in Example 1, Steps f) and g).

### EXAMPLE 5

### 9-(4-Methylthiazol-2-yl)-8-oxo-11-(pyridin-4-yl)-5,6,7,8-tetrahydro-7a-aza-2-azoniadibenzo[a,c]cyclohentene: bishydrochloride

Hydrochloric acid (0.85 ml, 1M in Et₂O, 0.85 mmol) was added to a hot solution of 9-(4-methylthiazol-2-yl)-11-(pyridin-4-yl)-6,7-dihydro-5H-2,7a-diazadibenzo[a,c]cyclohepten-8-one (160 mg, 0.41 mmol) in EtOH (25 ml) and CH₂Cl₂ (5 ml). The mixture was allowed to cool and upon addition of Et₂O a yellow solid crystallised. The solid was collected by filtration and washed with Et₂O and dried under vacuum to afford title salt (180 mg, 96%) as yellow plates, mp 304-307° C.: ¹H NMR (400 MHz, d₆-DMSO) δ 1.45-1.60 (m, 1H), 1.82-1.95 (m, 1H), 1.90 (s, 3H), 2.46-2.66 (m, 3H), 4.46 (dd, J--14 and 6, 1H), 6.96 (s, 1H), 7.16 (d, J=6, 2H), 7.46 (d, J=5, 1H), 7.77 (br s, 1H), 8.04 (d, J=6, 2H), 8.16 (br s, 2H), 8.46 (s, iH). m/z (ES⁺) 387 (M⁺+H).

1. 9-(4-Methylthiazol-2-yl)-11-(pyridin-4-yl)-6,7-dihydro-5H-2,7a-diazadibenzo[a,c]cyclohepten-8-one of formula I: 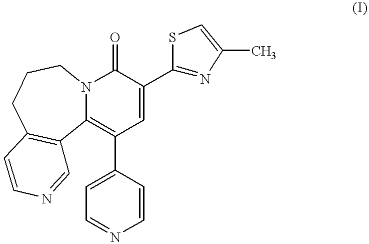
or a pharmaceutically acceptable salt thereof.

2. The bismethanesulfonate salt of 9-(4-methylthiazol-2-yl)-11-(pyridin-4-yl)-6,7-dihydro-5H-2,7a-diazadibenzo[a,c]cyclohepten-8-one.

3. The bishydrochloride salt of 9-(4-methylthiazol-2-yl) 11-(pyridin-4-yl)-6, 7-dihydro-5H-2, 7a-diazadibenzo [a,c]cyclohepten-8-one.

4. A pharmaceutical composition comprising the compound of formula I as depicted in claim 1 or a pharmaceutically acceptable salt thereof in combination with a pharmaceutically acceptable carrier.

5. A method for the treatment and/or prevention of anxiety which comprises administering to a patient in need of such treatment an effective amount of the compound of formula I as depicted in claim 1 or a pharmaceutically acceptable salt thereof.

6. A method for the treatment and/or prevention of convulsions which comprises administering to a patient in need of such treatment an effective amount of the compound of formula I as depicted in claim 1 or a pharmaceutically acceptable salt thereof.

7. A composition as claimed in claim 4 which is adapted for oral administration.

8. A process for the preparation of a compound as claimed in claim 1 which comprises:
(A) cyclising a compound of formula II: 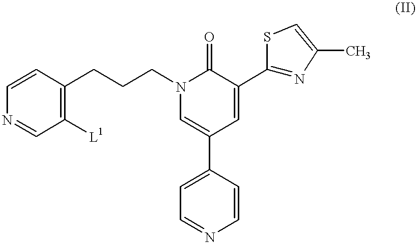
wherein L¹ represents a readily displaceable group; or
(B) cyclising a compound of formula III: 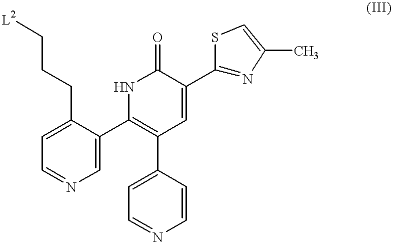
wherein L² represents a readily displaceable group; or
(C) reacting a compound of formula VI with a compound of formula VII: 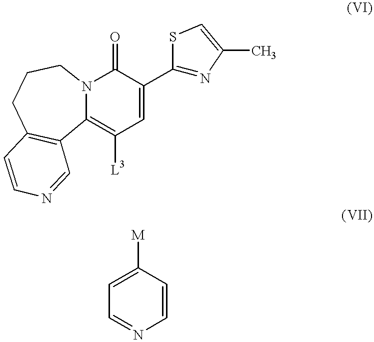
wherein M represents —B(OH)₂ or —Sn(Alk)₃ in which Alk represents a C₁₋₆ alkyl group, and L³ represents a suitable leaving group; in the presence of a transition metal catalyst.
